SEQUENCE LISTING (2 sequences)

SEQ ID NO: 1 - protein, 224 aa, Homo sapiens
MPGSPRPAPSWVLLLRLLALLRPPGLGEACSCAPAHPQQHICHSALVIRAKISSEKVVPASADPADTEKMLRYEIKQIKMFKGFEKVKDVQYIYTPFDSSLCGVKLEANSQKQYLLTGQVLSDGKVFIHLCNYIEPWEDLSLVQRESLNHHYHLNCGCQITTCYTVPCTISAPNECLWTDWLLERKLYGYQAQHYVCMKHVDGTCSWYRGHLPLRKEFVDIVQP

SEQ ID NO: 2 - DNA, 675 bp, Homo sapiens
atgcctgggagccctcggcccgcgccaagctgggtgctgttgctgcggctgctggcgttgctgcggcccccggggctgggtgaggcatgcagctgcgccccggcgcaccctcagcagcacatctgccactcggcacttgtgattcgggccaaaatctccagtgagaaggtagttccggccagtgcagaccctgctgacactgaaaaaatgctccggtatgaaatcaaacagataaagatgttcaaagggtttgagaaagtcaaggatgttcagtatatctatacgccttttgactcttccctctgtggtgtgaaactagaagccaacagccagaagcagtatctcttgactggtcaggtcctcagtgatggaaaagtcttcatccatctgtgcaactacatcgagccctgggaggacctgtccttggtgcagagggaaagtctgaatcatcactaccatctgaactgtggctgccaaatcaccacctgctacacagtaccctgtaccatctcggcccctaacgagtgcctctggacagactggctgttggaacgaaagctctatggttaccaggctcagcattatgtctgtatgaagcatgttgacggcacctgcagctggtaccggggccacctgcctctcaggaaggagtttgttgacatcgttcagccctag

(19) United States
(12) Patent Application Publication
(10) Pub. No.: US 2010/0047822 A1
(43) Pub. Date: Feb. 25, 2010

(54) TIMP-4 AS A BIOMARKER FOR THE DIAGNOSIS OF CARDIAC INSUFFICIENCY

(75) Inventors: Hendrik Milting, Bad Oeynhausen (DE); Frank Kramer, Wuppertal (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: BAYER SCHERING PHARMA AKTIENGESELLSCHAFT, Berlin (DE)

(21) Appl. No.: 12/594,549

(22) PCT Filed: Mar. 28, 2008
(86) PCT No.: PCT/EP2008/002463

§ 371 Date: Oct. 2, 2009

(30) Foreign Application Priority Data
Apr. 2, 2007 (DE) 10 2007 015 886.8

(51) Int. Cl. G01N 33/53

(52) U.S. Cl. 435/7.1

(57) ABSTRACT

The invention relates to the use of the plasma concentration of the polypeptide “tissue inhibitor of metalloproteinase-4” (TIMP-4) as a biomarker for the diagnosis of heart failure.

The invention relates to the use of the plasma concentration of “tissue inhibitor of metalloproteinase-4” (TIMP-4)¹ as a biomarker for the diagnosis of diastolic and systolic heart failure (DHF, SHF), right heart failure, left heart failure, and global heart failure (GHF) of acute or chronic intensity, and of the underlying cardiomyopathies, such as myocarditis and hypertrophic cardiomyopathy (HCM), dilatative cardiomyopathy (DCM), arrhythmogenic right ventricular cardiomyopathy (ARVCM), restrictive cardiomyopathy (RCM). The invention further relates to the diagnosis of right heart hypertrophy and right heart failure due to pulmonary artery hypertension (PAH), pulmonary hypertension in left heart disease, pulmonary hypertension associated with hypoxia, pulmonary hypertension due to chronically thrombotic and/or embolic diseases and pulmonary hypertension due to sarcoidosis, histiocytosis X, lymphangioleiomyomatosis.

The term “biomarkers for the diagnosis of” includes in this context in particular the following usages:
diagnosis of the aforementioned diseases and pathological changes
determination of the severity of the aforementioned diseases and pathological changes
monitoring of the effects of interventions, such as pharmacotherapy, surgery, use of medicinal products and measures accompanying therapy, on the aforementioned diseases and pathological changes
use as a surrogate marker, which replaces the determination of another parameter, which helps to assess the aforementioned diseases and pathological changes.

### Heart Failure

A number of factors can lead to development of the various forms of heart failure and their progression. Often events such as a local ischaemic attack (myocardial infarction) or viral myocarditis damage the heart muscle irreversibly and thus form the basis for development of the disease, but also insidious processes, such as persistent arterial hypertension or pulmonary artery hypertension lead to far-reaching changes in the homeostasis of the body. The increased volume load on the heart is compensated initially by myocardial hypertrophy, but finally leads to insidious damage of the cellular structures of the heart. As the most important pathological changes within the scope of these processes, we may mention neurohormonal changes², local and systemic inflammation , necrosis and apoptosis of cardiomyocytes^{4,5} and effects on renal electrolyte and water excretion⁶. The aforementioned damaging effects lead to activation of a gradual process of reconstruction of the heart tissue, the consequence of which is the deposition of collagenous structures in the myocardium and therefore has an adverse effect on various aspects of the functionality of the heart⁷. Interstitial fibrosis leads on the one hand to a stiffening of the myocardium and thus to restricted contractility, but on the other hand also hampers electrophysiological stimulus transport and therefore the excitation of the cardiomyocytes, which causes additional lowering of the pumping performance of the heart. As the final consequence, manifest heart failure is characterized by restricted pumping function of the heart, which leads to decreased blood flow and therefore inadequate supply of oxygen nutrients to the organs and in consequence physical fitness is restricted slightly to very considerably. Overall, heart failure is a progressive disease, which in addition to severe limitation of the patient's fitness and in some circumstances severe impairment of quality of life is associated with a very poor prognosis⁸.

For classification of the degrees of severity of heart failure, the proposal of the New York Heart Association (NYHA) for classifying patients in four classes has been widely adopted. Patients in the NYHA classes are characterized by:
NYHA I→Asymptomatic initial stage of heart failure, which can only be detected by special diagnostic methods.
NYHA II→Limitation of physical activity as a result of more severe physical exertion
NYHA III→The patient is asymptomatic when at rest, but there is limitation of physical activity as a result of normal physical exertion.
NYHA IV→The patient is unable to carry on any physical activity, as even at rest there are severe symptoms, so that as a rule bed rest is indicated.

The matrix metalloproteinases (MMPs) and their endogenous inhibitors the “tissue inhibitors of metalloproteinases” (TIMPs) play an important role in the structural reorganization of the heart tissue. Matrix metalloproteinases, which are also called matrixins, represent a family of Ca²⁺-dependent and Zn²⁺-dependent endopeptidases, which cleave a large number of proteins, in particular constituents of the extracellular matrix⁹. The local degradation of extracellular matrix is regulated by the expression of MMPs, by proteolytic activation of the MMP pro-enzymes and by deactivation of the MMPs based on binding of endogenous inhibitors, such as α2-macroglobulins or TIMPs^{10,11}. TIMPs bind non-covalently in a stoichiometric ratio of 1:1 to MMPs and thus block potential substrates' access to the catalytic domain.

Significantly increased MRNA and protein expression of MMP-2 and its inhibitor TIMP-1 has been observed in the hearts of HF patients^{12,13}. Furthermore, it was shown in various clinical studies that there is a correlation between the severity of heart failure and the patient's plasma MMP-2 and TIMP-1 concentrations¹⁴⁻¹⁷.

### Treatment of Heart Failure

Therapy for heart failure is directed essentially at
1. reducing the volume load on the heart
2. improving the energy balance of the heart
3. improving the contractility of the heart

These aims can be achieved with drug therapies, surgical intervention and measures accompanying therapy. The pharmacological, evidence-based therapy of chronic heart failure of NYHA classes I-IV is characterized by the use of inhibitors of the angiotensin-converting enzyme (ACE inhibitors), beta-receptor antagonists (β-blockers) and aldosterone antagonists. Diuretics, cardiac glycosides, angiotensin receptor antagonists, vasodilators and anticoagulants are also used, depending on symptoms and concomitant diseases¹⁸.

If the possibilities of pharmacotherapy do not result in stabilization of symptoms in patients with terminal failure, depending on the type of disease the last life-sustaining treatment option is implantation of pacemaker systems, supporting pumping systems, so-called “ventricular assist devices” (VAD) or heart transplant.

Biventricular Pacemakers

HF patients of NYHA classes III and IV with an ejection fraction of ≦35%, who additionally have ECG abnormalities such as non-synchronized contractions of the ventricles, benefit from implantation of a biventricular pacemaker in addition to pharmacotherapy. This resynchronization therapy leads as a rule to a marked improvement of symptoms and is therefore associated with a gain in quality of life. Furthermore, the implantation of a biventricular pacemaker system improves patients' prognosis and has been demonstrated to reduce their rehospitalization rate and mortality¹⁹.

Left Ventricular Assist Devices (LVAD)

In cases in which, even with pharmacotherapy and the use of other therapeutic measures, the heart does not produce adequate ejection performance and as a result the coronaries of the heart and the organs of the body are underperfused, there is the possibility of implanting battery-powered intra- or extracorporeal pumping systems, for mechanical support or replacement of heart function²⁰. Ideally the reduction in load brought about by the LVAD leads to normalization of gene expression in the tissues of the heart, abatement of local inflammatory processes, inhibition of active rearrangement processes of the working myocardium and thus enables the heart to regenerate. The support of cardiac function by the LVAD as a rule improves patients' overall condition to the extent that intensive medical care in hospital is no longer required²¹. In particular for patients who are on the heart transplant list and are waiting for a suitable donor heart, LVAD implantation represents an important life-sustaining measure²³. LVADs are fitted between the left ventricle and the aorta.

Transplantation

Heart transplantation or the implantation of an artificial heart represents the final option for preserving the life of patients with terminal heart failure, who are dependent on intensive medical care in hospital.

### Prior Art: Role of Biomarkers in the Diagnosis of Heart Failure

For the diagnosis of heart failure, a number of different methods are available, each of which is able to assess a particular functional parameter of the heart. In addition to methods that can already be carried out by the general practitioner, such as resting echocardiogram (echoCG) and exercise echoCG, more expensive differential-diagnostic imaging methods, such as magnetic resonance tomography (MRT) and cardiac catheterization coupled with angiography play an important role. In each case, biochemical assessment of various plasma parameters makes an important contribution to the diagnosis. Along with determination of the concentration of electrolytes and proinflammatory cytokines and chemokines, the detection of neurohumoral proteins is also very important. As representatives of this protein class, we may mention atrial natriuretic peptide (ANP) and B-type natriuretic peptide (BNP), which are currently the only recognized plasma markers for hospital assessment of heart failure. The BNP precursor hormone pre-proBNP is secreted by cardiomyocytes when they are subjected to an increased distension stimulus, such as occurs for example with increased volume load on the heart. In the circulation, the pre-proBNP is cleaved enzymatically into vasoactive BNP (C-terminus of pre-proBNP) and the biologically inactive NT-proBNP (N-terminus of pre-proBNP)²³. Both the plasma concentration of NT-proBNP and that of the biologically active BNP correlate very well with cardiac output. However, as the half-life of NT-proBNP is much greater than that of BNP, automated NT-proBNP analysis has been adopted in everyday hospital practice. For patients in all age groups, an NT-proBNP level of <300 pg/mL is regarded as an indicator that chronic heart failure can be ruled out with 83% probability. NT-proBNP concentrations above a limit of 900 pg/mL indicate, with 87% probability, the existence of chronic heart failure²⁴. However, as the BNP plasma concentration only provides information on the volume load on the heart, whereas the manifestation of heart failure is characterized decisively by fibrotic remodelling of the heart tissue, these neurohumoral markers only reflect a partial aspect of the disease. Moreover, the predictive value of the plasma BNP or NT-proBNP level is limited by the fact that increased release of natriuretic peptides also occurs in diseases other than heart failure. In particular in impairment of renal function to a glomerular filtration rate <60 mL/min, significantly increased BNP plasma concentrations occur²⁵. Moreover, non-alcohol-induced liver cirrhosis²⁶, intense physical loads²⁷, type I diabetes mellitus²⁸ and the infusion of quite large amounts of fluid lead to a rise in BNP level. Assessment of the BNP plasma concentration is additionally complicated by the fact that there is an inverse correlation between the “body mass index” (BMI) and the BNP or NT-proBNP plasma levels²⁹.

### Statement of the Problem

The invention is based on the problem of overcoming the aforementioned disadvantages and limitations of the prior art, in particular the limitations of diagnosis based on the BNP and NT-proBNP level, and of identifying a plasma marker that can be employed for the diagnosis of heart failure. Furthermore, the identified HF plasma marker should supplement, or replace in the sense of a surrogate marker, the information that can be obtained by an imaging or other kind of diagnostic method.

The problem was solved according to the invention, in that it was shown that it is possible to distinguish between healthy test subjects and patients with heart failure on the basis of the TIMP-4 plasma concentrations. Owing to the biological function of TIMP-4, TIMP-4 gives a better indication of the fibrotic remodelling of the heart than for example NT-proBNP and is unaffected by the volume load. This means for example that the diagnosis is not affected by previous infusion or diabetes. Moreover, in patients with heart failure the TIMP-4 plasma concentrations were correlated with functional parameters of the heart that had been acquired by imaging or other kinds of diagnostic methods.

The invention relates to methods of using TIMP-4 as a biomarker for the diagnosis of heart failure in a mammal, comprising the following stages:
a) Preparation of a sample to be diagnosed from a mammal
b) Determination of the amount of TIMP-4 in the sample to be diagnosed
c) Preparation of a control sample
d) Determination of the amount of TIMP-4 in the control sample
e) Comparison of the amount of TIMP-4 in the sample to be diagnosed with the control sample
f) Diagnosis of heart failure based on comparison of the amount of TIMP-4 in the sample to be diagnosed in comparison with the control sample.

The invention also relates to methods of using TIMP-4 as a biomarker for the diagnosis of heart failure in a mammal, comprising the following stages:
a) Obtaining a sample to be diagnosed from a mammal
b) Determination of the amount of TIMP-4 in the sample to be diagnosed
c) Comparison of the amount of TIMP-4 in the sample to be diagnosed with the control sample
d) Diagnosis of heart failure based on comparison of the amount of TIMP-4 in the sample to be diagnosed in comparison with the control sample.

The invention additionally relates to methods of using TIMP-4 as a biomarker for the diagnosis of heart failure in a mammal, comprising the following stages:
a) Determination of the amount of TIMP-4 in the sample to be diagnosed obtained from a mammal
b) Determination of the amount of TIMP-4 in the control sample obtained from a mammal
c) Comparison of the amount of TIMP-4 in the sample to be diagnosed with the control sample
d) Diagnosis of heart failure based on comparison of the amount of TIMP-4 in the sample to be diagnosed in comparison with the control sample.

The invention also relates to methods of using TIMP-4 as a biomarker for the diagnosis of heart failure in a mammal, comprising the following stages:
a) Determination of the amount of TIMP-4 in the sample to be diagnosed obtained from a mammal
b) Comparison of the amount of TIMP-4 in the sample to be diagnosed with the control sample
c) Diagnosis of heart failure based on comparison of the amount of TIMP-4 in the sample to be diagnosed in comparison with the control sample.

The method according to the invention describes the use of TIMP-4 as a biomarker for the diagnosis of heart failure. The use of TIMP-4 as a diagnostic biomarker is based on comparison of the amount of TIMP-4 in a sample to be diagnosed with the amount of TIMP-4 in a control sample or a TIMP-4 control value. Heart failure is diagnosed by comparing the amount of TIMP-4 in the sample to be diagnosed and the amount of TIMP-4 in the control sample. A difference in comparison of the TIMP-4 control value (obtained from the control sample) and the amount of TIMP-4 from the sample to be diagnosed permits the diagnosis of heart failure. The amount of TIMP-4 can be compared on the basis of the relative or absolute amount of TIMP-4. The absolute amount of TIMP-4 can be determined by means of a standard calibration curve, for which known amounts of TIMP-4 are used. A person skilled in the art knows from the prior art how to carry out such experiments.

In a preferred embodiment, heart failure is diagnosed if the amount of TIMP-4 in the sample to be diagnosed is higher than in the control sample, an increase by a factor of at least 1.5; 2; 2.5; 5; 7.5 or >10 being especially preferred.

The sample to be diagnosed and the control sample are obtained from a mammal. In a preferred embodiment of the invention the sample to be diagnosed and the control sample consist of a body fluid, blood being especially preferred, or samples obtained therefrom, such as serum or plasma.

In an especially preferred embodiment the sample to be diagnosed and the control sample are from a human being. The sample to be diagnosed and the control sample are obtained from analogous tissues, or body fluids.

In a preferred embodiment the normal or control values for TIMP-4 are obtained using the control sample that originates from healthy mammals. The control sample can be obtained by collecting several separate or even combined samples from mammals. A person skilled in the art knows how many samples must be used in order to achieve a statistically significant result. In another embodiment the control sample can be taken at an earlier time point or the amount of TIMP-4 can be determined in one or more control samples or the TIMP-4 control value can be determined at an earlier time point and for example stored in a database, an internet publication, in a manner permitting electronic acquisition or in a publication.

In an especially preferred embodiment the amount of TIMP-4 is determined via the amount of TIMP-4 polypeptide in the sample. A person skilled in the art knows methods from the prior art, for detecting polypeptides quantitatively. Examples are antibody-based methods such as ELISA, RIA or Western Blot. Another method is for example Planar Wave Guide Technology. A person skilled in the art knows methods from the prior art for producing anti-TIMP-4 antibodies. Wherein a TIMP-4 polypeptide is selected from a group comprising a polypeptide with the sequence of SEQ ID NO:1, a polypeptide that includes the sequence of SEQ ID NO:1, a polypeptide that is encoded by a TIMP-4 polynucleotide (SEQ ID NO:2), and polypeptides that have at least 99%, 98%, 95%, 90%, 88% or 80% identity with one of the aforementioned TIMP-4 polypeptides. The TIMP-4 polypeptide sequence is described in reference¹.

In another embodiment the amount of TIMP-4 is determined via the amount of TIMP-4 polynucleotide in the sample. A person skilled in the art knows methods from the prior art for detecting polynucleotides quantitatively. Examples are Northern blot, real-time PCR or TaqMan. Another method is for example Planar Wave Guide Technology.

The method according to the invention is especially preferred for the diagnosis of heart failure selected from the group comprising diastolic and systolic heart failure (DHF, SHF), right heart failure, left heart failure, and global heart failure (GHF) of acute or chronic intensity, and of the underlying cardiomyopathies, such as myocarditis and hypertrophic cardiomyopathy (HCM), dilatative cardiomyopathy (DCM), arrhythmogenic right ventricular cardiomyopathy (ARVCM), restrictive cardiomyopathy (RCM) and right heart hypertrophy and right heart failure due to pulmonary artery hypertension (PAH), pulmonary hypertension in left heart disease, pulmonary hypertension associated with hypoxia, pulmonary hypertension due to chronically thrombotic and/or embolic diseases and pulmonary hypertension due to sarcoidosis, histiocytosis X, lymphangioleiomyomatosis.

In a preferred embodiment the method according to the invention is used for the diagnosis of the aforementioned diseases and pathological changes.

In another preferred embodiment the method according to the invention is used for the diagnosis of the severity of the aforementioned diseases and pathological changes. In this embodiment it is possible to determine, as the TIMP-4 control sample or as the TIMP-4 control value, the amount of TIMP-4 in sick mammals, which are classified in defined groups according to particular criteria, for example the criteria of the NYHA.

In another preferred embodiment TIMP-4 is used as a therapy-accompanying biomarker in the method according to the invention for the diagnosis and monitoring of the effects of interventions, such as pharmacotherapy, surgery, the use of medicinal products and measures accompanying therapy for the aforementioned diseases and pathological changes. In this embodiment a sample to be diagnosed can be taken several times, i.e. in the course of therapy, and analysed by the method according to the invention. If the amount of TIMP-4 does not change, or only changes insignificantly, during a therapy, the therapy can be regarded as unsuccessful. If this is the case, repeat therapy with another dosage, for example an increased dose, or another dosage form can be considered. Moreover, an additional or another therapy can be considered. The method according to the invention can also be used in clinical studies for diagnosing the outcome of a therapy. This object of the invention comprises methods of using TIMP-4 as a biomarker for the diagnosis of the efficacy of a therapy for heart failure in a mammal, comprising the following stages:
a) Administration of a therapy for heart failure
b) Obtaining one or more further samples to be diagnosed from the treated mammal
c) Determination of the amount of TIMP-4 in the samples to be diagnosed
d) Preparation of a control sample
e) Determination of the amount of TIMP-4 in the control sample
f) Comparison of the amount of TIMP-4 in the samples to be diagnosed with the control sample
g) Diagnosis of the therapeutic outcome based on comparison of the amount of TIMP-4 in the samples to be diagnosed in comparison with the control sample.
The invention also relates to methods of using TIMP-4 as a biomarker for the diagnosis of the efficacy of a therapy for heart failure in a mammal, comprising the following stages:
a) Administration of a therapy for heart failure
b) Obtaining one or more further samples to be diagnosed from the treated mammal
c) Determination of the amount of TIMP-4 in the samples to be diagnosed
d) Comparison of the amount of TIMP-4 in the samples to be diagnosed with the control sample
e) Diagnosis of the therapeutic outcome based on comparison of the amount of TIMP-4 in the samples to be diagnosed in comparison with the control sample.

The invention also relates to methods of using TIMP-4 as a biomarker for the diagnosis of the efficacy of a therapy for heart failure in a mammal, comprising the following stages:
a) Determination of the amount of TIMP-4 in one or more samples to be diagnosed obtained from a mammal, which has received a therapy for heart failure
b) Comparison of the amount of TIMP-4 in the samples to be diagnosed with the control sample
c) Diagnosis of the therapeutic outcome based on comparison of the amount of TIMP-4 in the samples to be diagnosed in comparison with the control sample.

In another preferred embodiment the method according to the invention can be used as a surrogate marker, which replaces the determination of another parameter, which helps to assess the aforementioned disease and pathological changes.
In another embodiment the method according to the invention can be combined with one or more known clinical markers and methods of diagnosis selected from the group comprising blood pressure, heart rate, systemic vascular resistance, fractional shortening (FS), end-diastolic diameter (EDD), end-systolic diameter (ESD), ejection fraction (EF), left ventricular contractility (LVC), systolic pulmonary artery pressure (PAP_{sys}), diastolic pulmonary artery pressure (PAP_{dia}), left ventricular end-diastolic pressure (LVEDP), six-minute walk test, spiroergometry, metabolites, BNP, CRP, ANP, electrocardiogram (ECG), positron emission tomography (PET), computed tomography (CT), magnetic resonance tomography (MRT), functional magnetic resonance tomography (fMRT), diffusion tensor imaging (DT-MRT), echocardiography, ultrasound and impedance cardiography.

### FIGURES

FIG. 1:

The TIMP-4 concentration (c_{TIMP-4}) (Y=c_{TIMP-4} [ng/mL]) in serum samples from patients whose NT-pro-BNP was below a limit of 1000 pg/mL (Group A) was compared with the TIMP-4 concentrations of patients with raised NT-pro-BNP levels (>1000 pg/mL) (Group B). The black box represents 50% of all individual values (25th-75th percentile), the white bar indicates the median, the end of the vertical line shows the lowest and highest measured value. (A: Group A; B: Group B; Y: TIMP-4 concentration [ng/mL])

FIG. 2:

The NT-proBNP concentration (c_{NT-proBNP}) (Y=c_{NTproBNP} [pg/mL]) of the two groups of samples that were employed for determining the TIMP-4 levels (FIG. 1), are shown. The black box represents 50% of all individual values (25th-75th percentile), the white bar indicates the median and the end of the vertical line shows the lowest and highest measured value. (A: Group A; B: Group B; Y: NT-proBNP concentration [pg/mL])

FIG. 3:

The TIMP-4 concentration (c_{TIMP-4}) (Y=c_{TIMP-4} [ng/mL]) in serum samples from test subjects without heart failure (Group C) and from patients with terminal heart failure before LVAD implantation (Group D) is shown. The black box represents 50% of all individual values (25th-75th percentile), the white bar indicates the median and the end of the vertical line shows the lowest and highest measured value. (C: Group C; D: Group D; Y: TIMP-4 concentration [ng/mL])

FIG. 4:

The TIMP-4 (c_{TIMP-4}) (Y=c_{TIMP-4} [ng/mL]) in serum samples from test subjects without heart failure (Group E) and from patients suffering from pulmonary hypertension-induced right heart failure (Group F) is shown. The black box represents 50% of all individual values (25th-75th percentile), the white bar indicates the median and the end of the vertical line shows the lowest and highest measured value. (E: Group E; F: Group F; Y: TIMP-4 concentration [ng/mL])

FIG. 5:

SEQ ID NO:1 Human TIMP-4 polypeptide sequence

FIG. 6:

SEQ ID NO:2 Human TIMP-4 polynucleotide sequence

FIG. 7:

Paired sequence comparison of the TIMP-4 polypeptides of different mammals. The table lists the results of a TIMP-4 polypeptide sequence comparison of various mammals (A: Homo sapiens; B: Pan troglodytes; C: Macaca mulatta; D: Bos taurus; E: Canis familiaris; F: Felis catus; G: Mus musculus; H: Rattus norvegicus; I: Oryctolagus cuniculus). The comparison is expressed as % identity. For example, the human sequence (A: Homo sapiens) shows an identity of 88.8% to the mouse sequence (G: Mus musculus).

FIG. 8:

Table of the measured values of Group A (serum samples from patients with NT-pro-BNP below a limit of 1000 pg/mL), of the measurements shown in FIG. 1 and 2 and measurements carried out in Example 1. Symbols A: sample number; B: concentration of NT-proBNP [pg/mL]; C: concentration of TIMP-4 [ng/mL].

FIG. 9:

Table of the measured values of Group B (serum samples from patients with NT-pro-BNP of >1000 pg/mL), of the measurements shown in FIG. 1 and 2 and carried out in Example 1. Symbols A: sample number; B: concentration of NT-proBNP [pg/mL]; C: concentration of TIMP-4 [ng/mL].

FIG. 10:

Table of the measured values of Group C (serum samples from test subjects without heart failure), the measurements shown in FIG. 3 and carried out in Example 2. Symbols A: patient number; B: concentration of TIMP-4 [ng/mL].

FIG. 11:

Table of the measured values of Group D (serum samples from patients with terminal heart failure before LVAD implantation), of the measurements shown in FIG. 3 and carried out in Example 2. Symbols A: patient number; B: concentration of TIMP-4 [ng/mL].

FIG. 12:

Table of the measured values of Group E (serum samples from test subjects without right heart failure), of the measurements shown in FIG. 4 and carried out in Example 3. Symbols A: patient number; B: concentration of TIMP-4 [ng/mL].

FIG. 13:

Table of the measured values of Group F (serum samples from patients with right heart failure and pulmonary hypertension (PAP>25 mmHg)), of the measurements shown in FIG. 4 and carried out in Example 3. Symbols A: patient number; B: concentration of TIMP-4 [ng/mL].

The invention is explained in more detail in the following examples:

### EXAMPLES

### Example 1

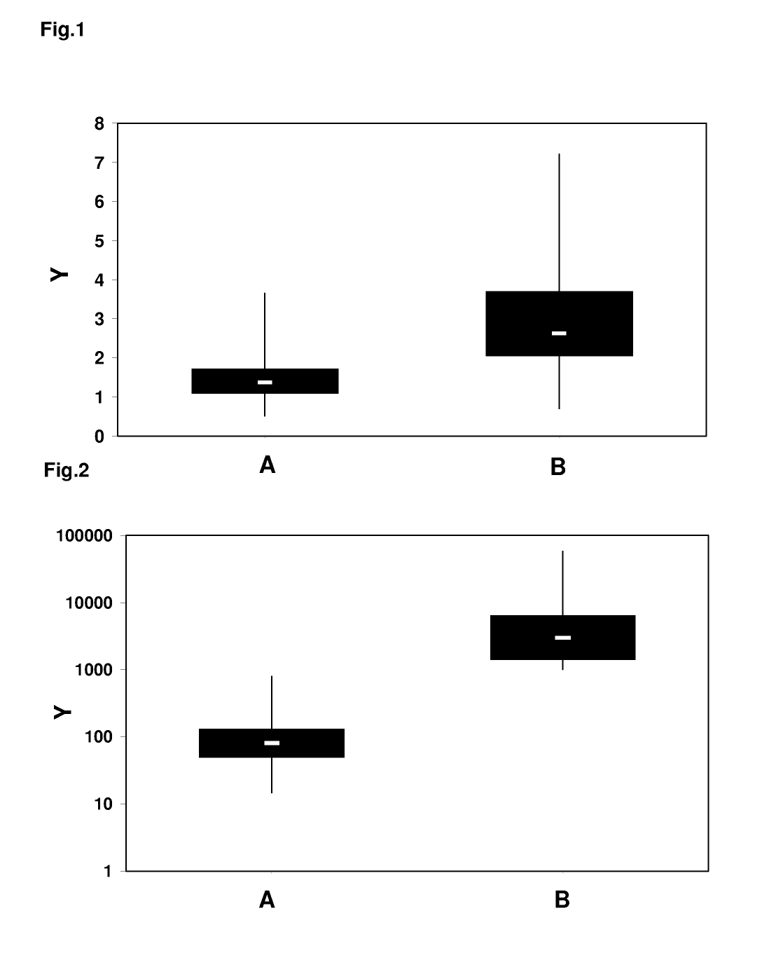

Owing to the high volume load on the heart, patients with heart failure are characterized by increased NT-proBNP plasma concentrations (see above). If the NT-proBNP concentration exceeds a value of 900 pg/mL, regardless of the patient's age and sex there is heart failure with 87% probability (see above). At NT-proBNP levels <300 pg/mL heart failure is ruled out with very high probability. Based on these relations, the TIMP-4 concentration was determined in serum samples from two groups of patients: 1) donors whose NT-proBNP level was between 15 and 795 pg/mL (median=81 pg/mL (no heart failure)). 2) donors whose NT-proBNP level was between 1016 and 58171 pg/mL (median=2976 pg/mL (high probability of heart failure)) (FIG. 2). The TIMP-4 serum concentration of the high-NT-proBNP group was increased highly significantly compared with the low-NT-proBNP group (FIG. 1) (low-NT-proBNP: median: 1.37 ng/mL, 25-75% percentile: 1.09-1.73 ng/mL, min.: 0.51 ng/mL, max.: 3.66 ng/mL; high-NT-proBNP: median: 2.62 ng/mL, 25-75% percentile: 2.04-3.70 ng/mL, min.:0.70 ng/mL, max.: 7.20 ng/mL; p<0.001).

### Example 2

Patients with terminal heart failure (NYHA IV), whose clinical situation has not improved under pharmacotherapy to the extent that intensive medical care has become unnecessary, and for whom in the short term no donor heart is available, are dependent on the implantation of a ventricular assist device (VAD) (see above).

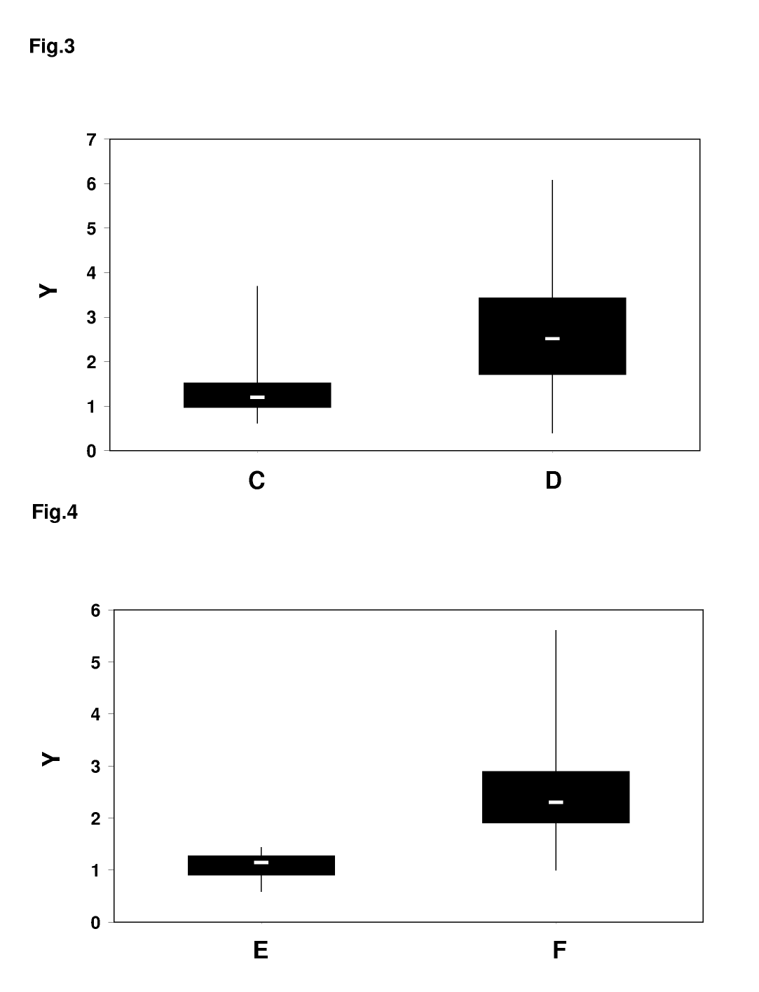

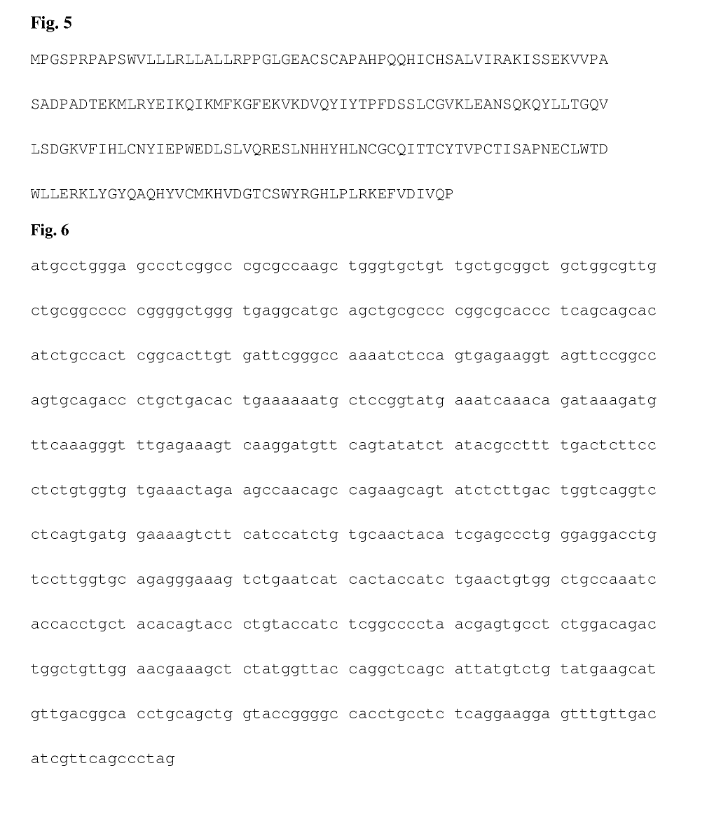

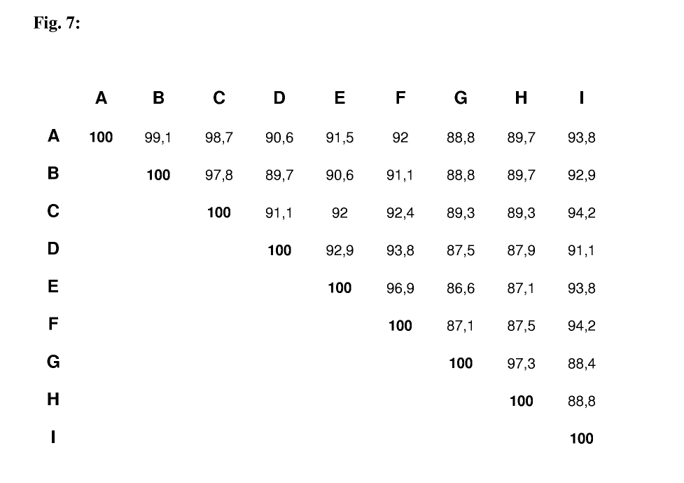

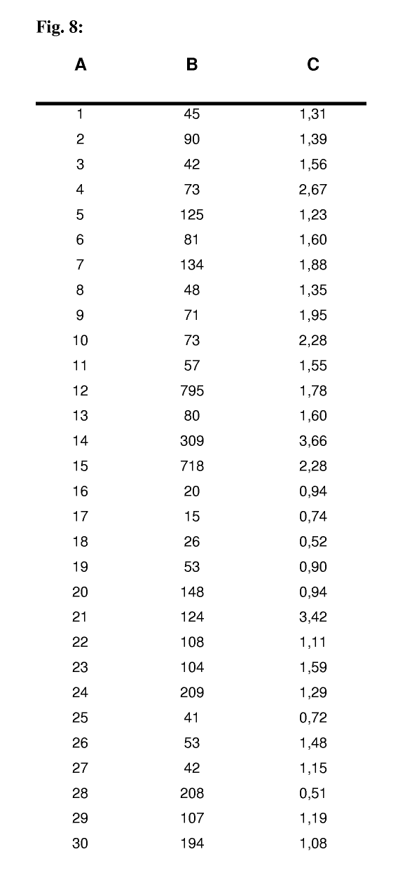

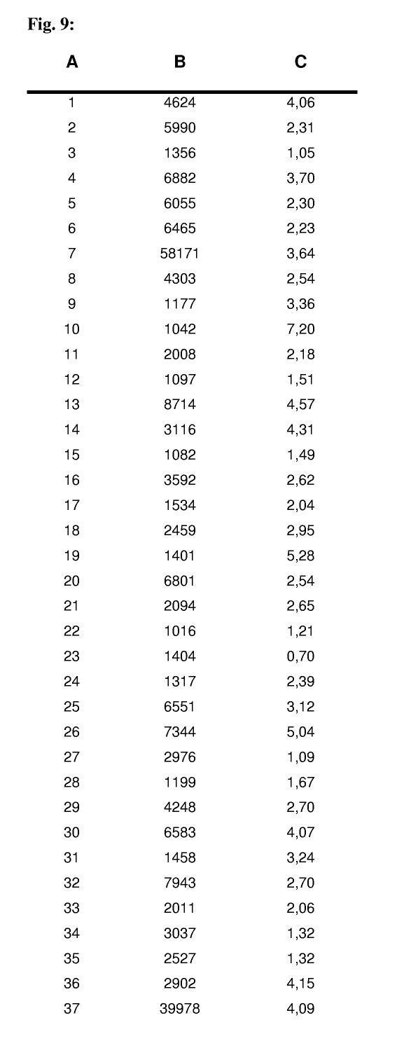

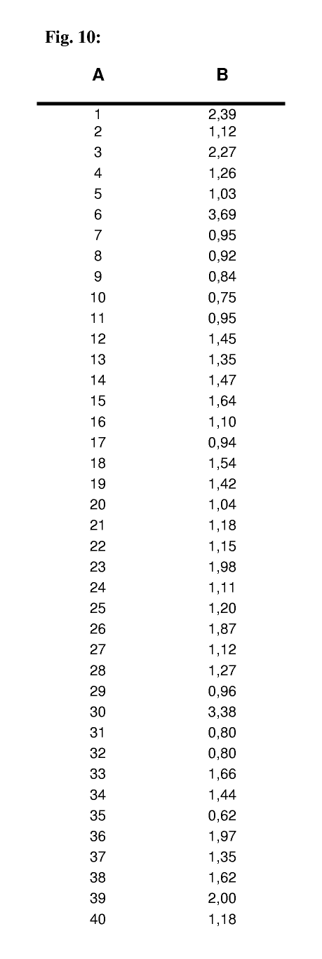

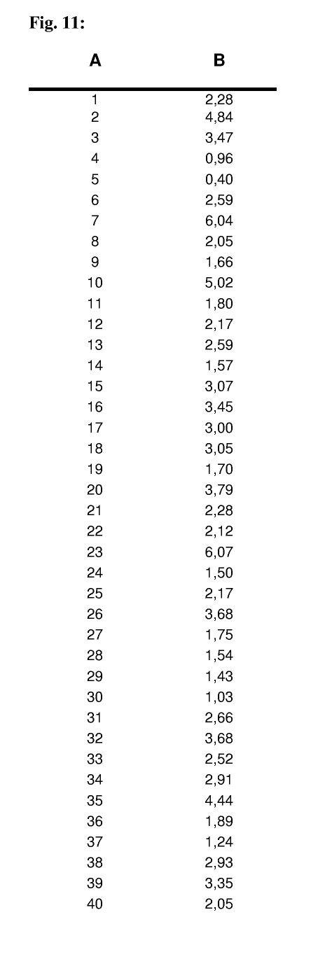

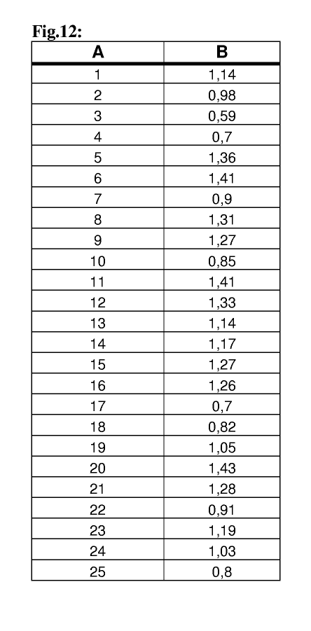

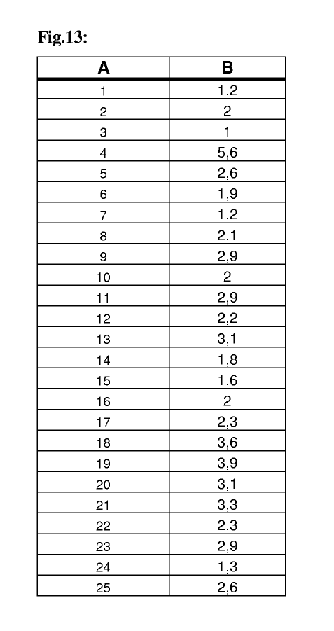

The TIMP-4 concentration in serum samples from patients who were on the list for implantation of a left ventricular assist device (LVAD) was compared with the TIMP-4 concentration in serum samples from healthy blood donors (FIG. 3). The TIMP-4 serum concentration in pre-LVAD patients was increased highly significantly compared with the healthy control group (control: median: 1.20 ng/mL, 25-75% percentile: 0.96-1.54 ng/mL, min.: 0.62 ng/mL, max.: 3.69 ng/mL; pre-LVAD: median: 2.52 ng/mL, 25-75% percentile: 1.70-3.45 ng/mL, min.: 0.40 ng/mL, max.: 6.07 ng/mL; p<0.001).

### Example 3

An important cause of right heart hypertrophy (cor pulmonale) and the resultant right ventricular failure is pulmonary hypertension, which is characterized by an increase in the resting pulmonary artery pressure (PAP) to values >25 mmHg (see above).

The TIMP-4 serum concentration of patients with right heart failure and pulmonary hypertension (RHI/PH) (n=25) was compared with the TIMP-4 serum concentration of healthy test subjects (n=25) (FIG. 4). The TIMP-4 serum concentration in the RHF/PH patients was increased highly significantly compared with the healthy control group (control: median: 1.14 ng/mL, 25-75% percentile: 0.90-1.28 ng/mL, min.: 0.59 ng/mL, max.: 1.43 ng/mL; RHF/PH: median: 2.30 ng/mL, 25-75% percentile: 1.90-2.90 ng/mL, min.: 1.0 ng/mL, max.: 5.60 ng/mL; p<0.001).

Materials and Methods

Sample Material

The TIMP-4 concentration in human serum samples was determined in two different studies.
1) The TIMP-4 concentration in serum samples from patients whose NT-proBNP was below a limit of 1000 pg/mL was compared with the TIMP-4 concentrations in patients with raised NT-pro-BNP levels (>1000 pg/mL). The NT-proBNP serum concentration was determined with the aid of the Elecsys® proBNP Chemiluminescence Immuno-Assay (Roche Diagnostics).
2) The TIMP-4 concentration in serum samples from healthy test subjects was compared with the TIMP-4 concentrations of patients with terminal heart failure, who were to receive an LVAD.

Reagents

The TIMP-4 concentration in the samples was determined using the human TIMP-4 Quantikine® ELISA from the company R&D Systems (Wiesbaden Nordenstadt, Germany; Catalogue No.: DTM400).

Protocol

The serum samples stored at −80° C. were thawed on ice, centrifuged for five minutes at 4° C. and 800 g and then added undiluted in accordance with the manufacturer's recommendations to concentration determination by ELISA.

Briefly, the ELISA protocol was characterized by the following steps:
Incubation of the samples in the 96-well microtitre plate coated with a monoclonal anti-human-TIMP-4 antibody
Removal of unbound sample constituents by washing the microtitre plate
Incubation of the microtitre plate with an HRP-conjugated secondary anti-human-TIMP-4 antibody
Removal of the excess HRP-conjugated secondary anti-human-TIMP-4 antibody by washing the microtitre plate
Incubation of the microtitre plate with TMB-enzyme substrate
Stopping the enzyme kinetics by adding dilute H₂SO₄ solution
Determination of the optical density (OD) of the wells of the microtitre plate at a wavelength of λ=450 nm (reference wavelength: λ=570 nm).
Calculation of the TIMP-4-calibration curve using the OD of the standard dilution series
Calculation of the sample TIMP-4 concentrations by plotting the sample OD against the OD of the standard samples
Statistical evaluation and graphical presentation of the measurement results (group mean values, standard deviation, “Standard error of the mean”, Student t-test)

Abbreviations
ANP=atrial natriuretic peptide
BNP=B-type natriuretic peptide
Ca²⁺=calcium ion
ECG=electrocardiogram
ELISA=enzyme linked immunosorbent assay
g=acceleration of gravity
HRP=horseradish peroxidase
H₂SO₄=sulphuric acid
LVAD=left ventricular assist device
λ=lambda (Greek)=wavelength
mL=millilitre
MMP matrix metalloproteinase
MRT=magnetic resonance tomography
nm=nanometre
NYHA=New York Heart Association
OD=optical density
PAP=pulmonary artery pressure
pg=picogram
TIMP=tissue inhibitor of metalloproteinases
TMB=tetramethylbenzidine
Zn²⁺=zinc ion

### REFERENCES

(1) Greene J, Wang M, Liu Y E, Raymond L A, Rosen C, Shi Y E. Molecular cloning and characterization of human tissue inhibitor of metalloproteinase 4. J Biol Chem 1996 Nov. 29; 271(48):30375-80.
(2) Bristow M R. The adrenergic nervous system in heart failure. N Engl J Med 1984 Sep. 27; 311(13):850-1.
(3) Dibbs Z, Kurrelmeyer K, Kalra D, Seta Y, Wang F, Bozkurt B, Baumgarten G, Sivasubramanian N, Mann D L. Cytokines in heart failure: pathogenetic mechanisms and potential treatment. Proc Assoc Am Physicians 1999 September; 111(5):423-8.
(4) Anversa P, Leri A, Beltrami C A, Guerra S, Kajstura J. Myocyte death and growth in the failing heart. Lab Invest 1998 July;78(7):767-86.
(5) Olivetti G, Abbi R, Quaini F, Kajstura S, Cheng W, Nitahara J A, Quaini E, Di L C, Beltrami C A, Krajewski S, Reed J C, Anversa P. Apoptosis in the failing human heart. N Engl J Med 1997 Apr. 17; 336(16).1131-41.
(6) Schrier R W. Body fluid volume regulation in health and disease: a unifying hypothesis. Ann Intern Med 1990 Jul. 15 ; 113(2):155-9.
(7) Cohn J N. Structural basis for heart failure. Ventricular remodeling and its pharmacologica inhibition. Circulation 1995 May 15; 91(10):2504-7.
(8) Bales A C, Sorrentino M J. Causes of congestive heart failure. Prompt diagnosis may affect prognosis. Postgrad Med 1997 January;101(1):44-6.
(9) Visse R, Nagase H. Matrix metalloproteinases and tissue inhibitors of metalloproteinases: structure, function, and biochemistry. Circ Res 2003 May 2; 92(8):827-39.
(10) Gomez D E, Alonso D F, Yoshiji H, Thorgeirsson U P. Tissue inhibitors of metalloproteinases: structure, regulation and biological functions. Eur J Cell Biol 1997 October;74(2):111-22.
(11) Bode W, Fernandez-Catalan C, Nagase R, Maskos K. Endoproteinase-protein inhibitor interactions. APMIS 1999 January;107(l):3-10.
(12) Polyakova V, Hein S, Kostin S, Ziegelhoeffer T, Schaper J. Matrix metalloproteinases and their tissue inhibitors in pressure-overloaded human myocardium during heart failure progression. J Am Coll Cardiol 2004 Oct. 19; 44(8):1609-18.
(13) Spinale F G. Matrix metalloproteinases: regulation and dysregulation in the failing heart. Circ Res 2002 Mar. 22; 90(5):520-30.
(14) Noji Y, Shimizu M, Ino H, Higashikata T, Yamaguchi M, Nohara A, Hiorita T, Shimizu K, Ito Y, Matsuda T, Namura M, Mabuchi H. Increased circulating matrix metalloproteinase-2 in patients with hypertrophic cardiomyopathy with systolic dysfunction. Circ J 2004 April;68(4):355-60.
(15) Ahmed S H, Clark L L, Pennington W R, Webb C S, Bonnema D D, Leonardi A H, McClure C D, Spinale F G, Zile M R. Matrix metalloproteinases/tissue inhibitors of metalloproteinases: relationship between changes in proteolytic determinants of matrix composition and structural, functional, and clinical manifestations of hypertensive heart disease. Circulation 2006 May 2; 113(17):2089-96.
(16) Yamazaki T, Lee J D, Shimizu H, Uzui H, Ueda T. Circulating matrix metalloproteinase-2 is elevated in patients with congestive heart failure. Eur J Heart Fail 2004 January;6(1):41-5.
(17) George J, Patal S, Wexler D, Roth A, Sheps D, Keren G. Circulating matrix metalloproteinase-2 but not matrix metalloproteinase-3, matrix metalloproteinase-9, or tissue inhibitor of metalloproteinase-1 predicts outcome in patients with congestive heart failure. Am Heart J 2005 September;150(3):484-7.
(18) Larson L W, Gerbert D A, Herman L M, Leger M M, MeNellis R, O'Donoghue D L, Ulshafer C, Van Dyke E M. ACC/AHA 2005 guideline update: chronic heart failure in the adult. JAAPA 2006 April; 19(4):53-6.
(19) Manolis A S. Cardiac resynchronization therapy in congestive heart failure: Ready for prime time? Heart Rhythm 2004 September;1(3):355-63.
(20) Long J W, Kfoury A G, Slaughter M S, Silver M, Milano C, Rogers J, Delgado R, Frazier O H. Long-term destination therapy with the HeartMate XVE left ventricular assist device: improved outcomes since the REMATCH study. Congest Heart Fail 2005 May;11(3):133-8.
(21) Entwistle J W, III. Short- and long-term mechanical ventricular assistance towards myocardial recovery. Surg Clin North Am 2004 February;84(l):201-21.
(22) Kherani A R, Oz M C. Ventricular assistance to bridge to transplantation. Surg Clin North Am 2004 February;84(1):75-ix.
(23) Wiviott S D, de Lemos J A, Morrow D A. Pathophysiology, prognostic significance and clinical utility of B-type natriuretic peptide in acute coronary syndromes. Clin Chim Acta 2004 Aug. 16; 346(2):119-28.
(24) Januzzi J L, Jr., Camargo C A, Anwaruddin S, Raggish A L, Chen A A, Krauser D G, Tung R, Cameron R, Nagurney J T, Chae C U, Lloyd-Jones D M, Brown D F, Foran-Melanson S, Sluss P M, Lee-Lewandrowski E, Lewandrowski K B. The N-terminal Pro-BNP investigation of dyspnea in the emergency department (PRIDE) study. Am J Cardiol 2005 Apr. 15; 95(8):948-54.
(25) Tsutamoto T, Wada A, Sakai H, Ishikawa C, Tanaka T, Hayashi M, Fujii M, Yamamoto T, Dohke T, Ohnishi M, Takashima H, Kinoshita M, Horie M. Relationship between renal function and plasma brain natriuretic peptide in patients with heart failure. J Am Coll Cardiol 2006 Feb. 7; 47(3):582-6.
(26) Yildiz R, Yildirim B, Karineaoglu M, Harputluoglu M, Hilmoglu F. Brain natriuretic peptide and severity of disease in non-alcoholic cirrhotic patients. J Gastroenterol Hepatol 2005 July;20(7):1115-20.
(27) Niessner A, Ziegler S, Slany J, Billensteiner E, Woloszczuk W, Geyer G. Increases in plasma levels of atrial and brain natriuretic peptides after running a marathon: are their effects partly counterbalanced by adrenocortical steroids? Eur J Endocrinol 2003 December; 149(6):555-9.
(28) McKenna K, Smith D, Sherlock M, Moore K, O'Brien E, Tormey W, Thompson C J. Elevated plasma concentrations of atrial and brain natiriuretic peptide in type 1 diabetic subjects. Ir J Med Sci 2005 July; 174(3):53-7.
(29) Krauser D G, Lloyd-Jones D M, Chae C U, Cameron R, Anwaruddin S, Baggish A L, Chen A, Tung R, Januzzi J L, Jr. Effect of body mass index on natriuretic peptide levels in patients with acute congestive heart failure: a ProBNP Investigation of Dyspnea in the Emergency Department (PRIDE) substudy. Am Heart J 2005 April;149(4):744-50.

1. Method of using TIMP-4 as a biomarker for the diagnosis of heart failure in a mammal, comprising the following steps:
a) Determination of the amount of TIMP-4 in the sample to be diagnosed obtained from a mammal,
b) Comparison of the amount of TIMP-4 in the sample to be diagnosed with a control sample
c) Diagnosis of heart failure based on comparison of the amount of TIMP-4 in the sample to be diagnosed in comparison with the control sample.

2. Method of using TIMP-4 as a biomarker for the diagnosis of heart failure in a mammal, comprising the following steps:
a) Obtaining a sample to be diagnosed from a mammal,
b) Determination of the amount of TIMP-4 in the sample to be diagnosed
c) Comparison of the amount of TIMP-4 in the sample to be diagnosed with a control sample
d) Diagnosis of heart failure based on comparison of the amount of TIMP-4 in the sample to be diagnosed in comparison with the control sample.

3. Method according to claims 1 and 2, characterized in that the sample is selected from the group comprising blood, serum and plasma.

4. Method according to claims 1 and 2, characterized in that the mammal is a human being.

5. Method according to claims 1 and 2, characterized in that the amount of TIMP-4 is determined on the basis of the amount of TIMP-4 polypeptide.

6. Method according to claims 1 and 2, characterized in that in the diagnosis of heart failure the amount of TIMP-4 in the sample to be diagnosed is increased in comparison with the control sample.

7. Method according to claims 1 and 2, characterized in that the use of TIMP-4 as a biomarker is combined with one or more other biomarkers and methods of diagnosis.

8. Method according to claims 1 and 2, characterized in that TIMP-4 is used as a therapy-accompanying biomarker.

9. Method according to claims 1 and 2, characterized in that TIMP-4 is used as a biomarker for determining the severity of heart failure.

10. Test kit for carrying out one of the methods according to claims 1 to 9 comprising at least one or more antibodies, which recognize TIMP-4 specifically, a detection means, one or more containers for collecting and storing the sample to be diagnosed.
